(19) United States
(12) Patent Application Publication
(10) Pub. No.: US 2010/0093773 A1
(43) Pub. Date: Apr. 15, 2010

(54) METHODS OF TREATING CANCER

(75) Inventors: MARK LAUGHLIN, Sunnyvale, CA (US); Mark B. Anderson, Oakland, CA (US); Adam Willardsen, Salt Lake City, UT (US); Chris Pleiman, Holladay, UT (US)

Correspondence Address: Myriad PHARMACEUTICALS, Inc.;c/o CPA Global, P.O. Box 52050, Minneapolis, MN, 55402, US

(73) Assignee: Myriad Pharmaceuticals, Inc., Salt Lake City, UT (US)

(21) Appl. No.: 12/574,632

(22) Filed: Oct. 6, 2009

Related U.S. Application Data
(63) Continuation of application No. PCT/US2008/059910, filed on Apr. 10, 2008.
(60) Provisional application No. 60/910,944, filed on Apr. 10, 2007.

(51) Int. Cl. A61K 31/517; A61P 35/00

(52) U.S. Cl. 514/266.4

(57) ABSTRACT

Disclosed is (4-Methoxy-phenyl)-methyl-(2-methyl-quinazolin-4-yl)-amine hydrochloride effective as a vascular disrupting agent. (4-Methoxy-phenyl)-methyl-(2-methyl-quinazolin-4-yl)-amine hydrochloride is useful in the treatment of a variety of clinical conditions in which uncontrolled growth and spread of abnormal cells occurs, and in particular to its use in treating cancer.

### CROSS REFERENCE TO RELATED APPLICATIONS

This application is a continuation of International Application No. PCT/US2008/059910, filed Apr. 10, 2008 and published as WO 2008/124826, which claims the benefit of U.S. Provisional Application Ser. No. 60/910,944, filed on Apr. 10, 2007, both of which are incorporated by reference herein in their entirety.

### FIELD OF THE INVENTION

This invention is in the field of medicinal chemistry. In particular, the invention relates to (4-Methoxy-phenyl)-methyl-(2-methyl-quinazolin-4-yl)-amine hydrochloride as a vascular disrupting agent and use as a therapeutically effective anti-cancer agent.

### BACKGROUND OF THE INVENTION

Cancer is a common cause of death in the world; about 10 million new cases occur each year, and cancer is responsible for 12% of deaths worldwide, making cancer the third leading cause of death. World Health Organization, National Cancer Control Programmes. Policies and Managerial Guidelines (2d ed. 2002).

The formation of new blood vessels, or angiogenesis, is an essential part of cancer. In the adult, angiogenesis is typically limited, occurring only in the process of wound healing and neovascularization of the endometrium during menstruation. See Merenmies et al., Cell Growth & Differentiation, 8, 3 10 (1997). In cancer, however, the new vessels allow tumor cells to grow and escape into the circulation and lodge in other organs. Accordingly, leading therapies for cancer include agents that disrupt the vascular system and/or angiogenesis.

Ideal chemotherapeutic agents would have specificity for cancer and tumor cells, while not affecting normal cells. Unfortunately, none have been found and instead agents that target the vascular system or rapidly dividing cells (both tumor and normal) are often used. Therefore, there remains a definite need in the art for the discovery of new effective chemotherapeutic agents that can be administered safely.

### SUMMARY OF THE INVENTION

The present invention is related to the discovery that (4-Methoxy-phenyl)-methyl-(2-methyl-quinazolin-4-yl)-amine hydrochloride is active as a vascular disrupting agent. Vascular disrupting agents and antiangiogenic compounds are known to be effective as combination therapy for cancer. Accordingly, an aspect of the present invention is directed to the use of (4-Methoxy-phenyl)-methyl-(2-methyl-quinazolin-4-yl)-amine hydrochloride in combination with one or more antiangiogenic compounds as therapy for cancer. In another aspect of the present invention, (4-Methoxy-phenyl)-methyl-(2-methyl-quinazolin-4-yl)-amine hydrochloride may be used as therapy for cancer in combination with a cytotoxic agent. In one embodiment, (4-Methoxy-phenyl)-methyl-(2-methyl-quinazolin-4-yl)-amine hydrochloride is administered as therapy for cancer at a dose of not more than about 4.5 mg/m². For example, the invention provides a method for treating cancer at a dose of between about 0.3 to about 3.3 mg/m², such as 2.1 mg/m² and 3.3 mg/m². In particular examples, (4-Methoxy-phenyl)-methyl-(2-methyl-quinazolin-4-yl)-amine hydrochloride may be administered for treatment of cancer at a dose of between about 0.5 mg to about 15 mg, such as about 2 mg to about 10 mg, or about 4 mg to about 8 mg.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

It is known that (4-Methoxy-phenyl)-methyl-(2-methyl-quinazolin-4-yl)-amine hydrochloride, is active as a potent tubulin inhibitor and cytotoxic agent. Here, it has been discovered that (4-Methoxy-phenyl)-methyl-(2-methyl-quinazolin-4-yl)-amine hydrochloride is a vascular disrupting agent. Vascular disrupting agents target the tumor neovascular endothelium leading to disruption of tumor blood supply and subsequent tumor cell necrosis. See Lippert III J., Biorg Med Chem 2007, 15:605-615; and Siemann et al., Clin Cancer Res. 2005, 11:416-420. Vascular disrupting agents in combination with antiangiogenic compounds are known to be effective therapy for cancer. Siemann and Shi, Int. J. Radiat. Oncol. Biol. Phys., 2004 60:1233-1240. Accordingly, an aspect of the present invention is directed to the use of (4-Methoxy-phenyl)-methyl-(2-methyl-quinazolin-4-yl)-amine hydrochloride in combination with one or more chemotherapeutic agents having an antiangiogenic effect as therapy or prophylaxis for cancer, which is a group of diseases characterized by the uncontrolled growth and spread of abnormal cells.

Such diseases include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

In one embodiment, (4-Methoxy-phenyl)-methyl-(2-methyl-quinazolin-4-yl)-amine hydrochloride is administered as therapy for cancer at a dose of not more than about 4.5 mg/m². In certain embodiments, (4-Methoxy-phenyl)-methyl-(2-methyl-quinazolin-4-yl)-amine hydrochloride is administered at a dose of not more than about 3.3 mg/m². In some embodiments, (4-Methoxy-phenyl)-methyl-(2-methyl-quinazolin-4-yl)-amine hydrochloride is administered at a dose of not more than about 2.7 mg/m². In further embodiments, (4-Methoxy-phenyl)-methyl-(2-methyl-quinazolin-4-yl)-amine hydrochloride is administered at a dose of not more than about 2.1 mg/m². For example, the invention provides a method for treating cancer at a dose of between about 0.3 to about 3.3 mg/m², such as between about 2.1 mg/m² and about 3.3 mg/m².

For example, (4-Methoxy-phenyl)-methyl-(2-methyl-quinazolin-4-yl)-amine hydrochloride may be administered for treatment of cancer at a dose of between about 0.5 mg to about 15 mg, such as about 2 mg to about 10 mg, or about 4 mg to about 8 mg. In certain embodiments, 4-Methoxy-phenyl)-methyl-(2-methyl-quinazolin-4-yl)-amine hydrochloride is administered at a dose of not more than about 10 mg, such as not more than about 8 mg or not more than about 6 mg. In additional embodiments, 4-Methoxy-phenyl)-methyl-(2-methyl-quinazolin-4-yl)-amine hydrochloride is administered at a dose of about 2, about 3, about 4, about 5, about 6, about 7, or about 8 mg.

While not wishing to be bound by any one theory, it is thought that (4-Methoxy-phenyl)-methyl-(2-methyl-quinazolin-4-yl)-amine hydrochloride and an antiangiogenic agent may act synergistically in the treatment of cancer. Accordingly, smaller dosages may be used than what is typically administered. Therefore, in some embodiments (4-Methoxy-phenyl)-methyl-(2-methyl-quinazolin-4-yl)-amine hydrochloride may be administered at a dose of not more than about 2.5 mg/m², such as not more than about 1.5 mg/m², or not more than about 0.5 mg/m². For example, in some embodiments (4-Methoxy-phenyl)-methyl-(2-methyl-quinazolin-4-yl)-amine hydrochloride may be administered at a dose of not more than about 4 mg, such as not more than about 3 mg, not more than about 2 mg, or not more than about 1 mg.

Chemotherapeutic agents having an antiangiogenic effect (an “antiangiogenic agent”) are determined according to the assay described in Example 10. Antiangiogenic agents include agents such as vascular endothelial growth factor (VEGF) inhibitors and agents that chelate or reduce the level of copper. Examples of VEGF inhibitors include Avastin® (bevacizumab), Sutent® (sunitinib), Nexavar® (sorafenib), vatalanib, semaxanib, ZD6474, SU6668, AG-013736, AZD2171, and AEE788. In certain embodiments, VEGF inhibitors useful in the present invention are chosen from bevacizumab, sunitinib, and sorafenib.

In some embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with an antiangiogenic agent. In some embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with an antiangiogenic agent chosen from Avastin® (bevacizumab), Sutent® (sunitinib), Nexavar® (sorafenib), vatalanib, semaxanib, ZD6474, SU6668, AG-013736, AZD2171, and AEE788. In certain embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with an antiangiogenic agent chosen from bevacizumab, sunitinib, and sorafenib.

For example (4-Methoxy-phenyl)-methyl-(2-methyl-quinazolin-4-yl)-amine hydrochloride may be administered at a dose of between about 0.5 mg to about 15 mg, such as about 2 mg to about 10 mg, or about 4 mg to about 8 mg in combination with one an antiangiogenic agent, such as an antiangiogenic agent chosen from Avastin® (bevacizumab), Sutent® (sunitinib), Nexavar® (sorafenib), vatalanib, semaxanib, ZD6474, SU6668, AG-013736, AZD2171, and AEE788. In certain embodiments, 4-Methoxy-phenyl)-methyl-(2-methyl-quinazolin-4-yl)-amine hydrochloride is administered at a dose of not more than about 10 mg, such as not more than about 8 mg or not more than about 6 mg in combination with an antiangiogenic agent chosen from bevacizumab, sunitinib, and sorafenib.

Agents that chelate or reduce the level of copper are also known to inhibit angiogenesis. Angiogenesis requires copper, as has been shown by numerous studies (Parke et al., Am. J. Pathol. 1988, 137:173 178; Raju et al., Natl. Cancer Inst. 1982, 69: 1183 1188; Ziche et al., Natl. Cancer Inst. 1982, 69: 475 482; Gullino, Anticancer Res. 1986, 6(2): 153 158). Attempts at preventing angiogenesis and hence tumor growth in animal models by reducing in vivo amounts of copper have been reported in the art (Brem et al., Neurosurgery 1990, 26:391 396; Brem et al., Am. J. Pathol. 1990, 137(5): 1121 1142; Yoshida et al., Neurosurgery 1995 37(2): 287 295). These approaches incorporated both copper chelators and low copper diets. More recently, Brewer et al., International Application No. PCT/US99/20374 have shown that the copper chelators, (e.g., tetrathiomolybdate) may be effective in treating diseases (e.g., solid tumor growth), which require angiogenesis.

In addition to the induction of angiogenesis, copper may also have a direct role in tumor cell growth and survival. High copper levels exist in both the plasma and in tumor tissue from patients with many different solid cancers (Chakravarty et al., J Cancer Res. Clin. Oncol. 1984, 108: 312 315). Recently, tetrathiomolybdate has been shown to down-regulate the expression of NF-.kappa.B as well as inhibit its translocation to the nucleus in the inflammatory breast cancer cell line SUM 149 (Pan et al., Cancer Res. 2002, 62: 4854 4859). The NF-.kappa.B system may be involved in mediating tumor cell survival and thus its down-regulation in tumor cells by tetrathiomolybdate suggests a direct effect of copper chelation on tumor survival.

Copper is both a requirement and a potent stimulus for angiogenesis, as shown by studies of neovascularization in the rabbit cornea (Parke et al., 1988). During prostaglandin E1 (PGE1)-induced angiogenesis in the rabbit cornea, copper accumulates at the site where angiogenesis occurs (Parke et al., 1988). Conversely, in copper deficient rabbits, angiogenesis in the rabbit cornea in response to PGE1 is greatly reduced. In the rabbit cornea, copper for angiogenesis can be supplied by ceruloplasmin (a copper protein) as well as by dissolved copper sulfate, while apoceruloplasmin (ceruloplasmin without copper) does not support angiogenesis (Gullino, 1986). Additional studies have also shown that copper is an important angiogenic agent (Raju et al., 1982; Ziche et al., 1982). These studies all support the concept that unbound copper is required for angiogenesis.

Examples of copper chelating agents include Cuprimine® (penicillamine, depen), Coprexa™ (Tetrathiomolybdate), syprine (trientine), BAL (British Anti-Lewisite, DMPS, Dimercaprol, dimercaptopropanol, Unithiol), DMSA (dimercaptosuccinic acid, DMS, Chemet), clioquinol, pyrrolidine dithiocarbamate, alpha-lipoic acid (ALA), L-taurine, pyrrolidine dithiocarbamate (PDTC), brucillamine, and NSAIDs. In particular embodiments, (4-Methoxy-phenyl)-methyl-(2-methyl-quinazolin-4-yl)-amine hydrochloride may be administered with a VEGF inhibitor, such as bevacizumab. In particular embodiments, (4-Methoxy-phenyl)-methyl-(2-methyl-quinazolin-4-yl)-amine hydrochloride may be administered with a copper chelating agent, such as penicillamine, trientine, and/or tetrathiomolybdate.

In some embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with a copper chelating agent. In some embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with a copper chelating agent chosen from Cuprimine® (penicillamine, depen), Coprexa™ (Tetrathiomolybdate), syprine (trientine), BAL (British Anti-Lewisite, DMPS, Dimercaprol, dimercaptopropanol, Unithiol), DMSA (dimercaptosuccinic acid, DMS, Chemet), clioquinol, pyrrolidine dithiocarbamate, alpha-lipoic acid (ALA), L-taurine, pyrrolidine dithiocarbamate (PDTC), brucillamine, and NSAIDs. In certain embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with a copper chelating agent chosen from penicillamine, trientine, and/or tetrathiomolybdate.

In another aspect of the present invention, (4-Methoxy-phenyl)-methyl-(2-methyl-quinazolin-4-yl)-amine hydrochloride may be used as therapy for cancer in combination with a cytotoxic agent. Cytotoxic agents useful in the present invention include nitrosoureas, such as Temodar® (temozolomide), dacarbazine, BCNU, and CCNU; taxanes, such as paclitaxel and docetaxel; vinka alkaloids, such as vincristine, vinblastine, and vinorelbine; topoisomerase inhibitors, such as etoposide, teniposide, Hycamtin® (topotecan), and Camptosar® (irinotecan); anthracyclines, such as doxorubicin, daunomycin, epirubicin, and idarubicin; antimetabolites, such as methotrexate, fluorouracil, cytarabine, Gemzar® (gemcitibine), and capecitibine; and platinum agents, such as cisplatin, carboplatin, and Eloxatin® (oxaliplatin). In certain embodiments, (4-Methoxy-phenyl)-methyl-(2-methyl-quinazolin-4-yl)-amine hydrochloride may be used as therapy for cancer in combination with one or more agents chosen from temozolomide, dacarbazine, BCNU, CCNU, vinorelbine, teniposide, irinotecan, daunomycin, idarubicin, cytarabine, gemcitibine, capecitibine, and oxaliplatin.

In some embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with a cytotoxic agent. In some embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with a cytotoxic agent chosen from temozolomide, dacarbazine, BCNU, CCNU, paclitaxel, docetaxel, vincristine, vinblastine, vinorelbine, etoposide, teniposide, topotecan, irinotecan, doxorubicin, daunomycin, epirubicin, idarubicin, methotrexate, fluorouracil, cytarabine, gemcitibine, capecitibine, cisplatin, carboplatin, and oxaliplatin. In certain embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with a cytotoxic agent chosen from temozolomide, dacarbazine, BCNU, CCNU, vinorelbine, teniposide, irinotecan, daunomycin, idarubicin, cytarabine, gemcitibine, capecitibine, and oxaliplatin. In certain embodiments, 4-Methoxy-phenyl)-methyl-(2-methyl-quinazolin-4-yl)-amine hydrochloride is administered at a dose of not more than about 4.5 mg/m², such as not more than about 3.3 mg/m² or not more than about 2.1 mg/m² in combination with carboplatin. In a particular embodiment, (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with oxaliplatin.

For example (4-Methoxy-phenyl)-methyl-(2-methyl-quinazolin-4-yl)-amine hydrochloride may be administered at a dose of between about 0.5 mg to about 15 mg, such as about 2 mg to about 10 mg, or about 4 mg to about 8 mg in combination with one a cytotoxic agent, such asa cytotoxic agent chosen from temozolomide, dacarbazine, BCNU, CCNU, paclitaxel, docetaxel, vincristine, vinblastine, vinorelbine, etoposide, teniposide, topotecan, irinotecan, doxorubicin, daunomycin, epirubicin, idarubicin, methotrexate, fluorouracil, cytarabine, gemcitibine, capecitibine, cisplatin, carboplatin, and oxaliplatin. In certain embodiments, 4-Methoxy-phenyl)-methyl-(2-methyl-quinazolin-4-yl)-amine hydrochloride is administered at a dose of not more than about 10 mg, such as not more than about 8 mg or not more than about 6 mg in combination with carboplatin.

In practicing the methods of the present invention, (4-Methoxy-phenyl)-methyl-(2-methyl-quinazolin-4-yl)-amine hydrochloride may be administered together with at least one known chemotherapeutic agent as part of a unitary pharmaceutical composition. Alternatively, (4-Methoxy-phenyl)-methyl-(2-methyl-quinazolin-4-yl)-amine hydrochloride may be administered apart from at least one known cancer chemotherapeutic agent. In one embodiment, (4-Methoxy-phenyl)-methyl-(2-methyl-quinazolin-4-yl)-amine hydrochloride and at least one known cancer chemotherapeutic agent are administered substantially simultaneously, i.e. the compounds are administered at the same time or one after the other, so long as the compounds reach therapeutic levels in the blood at the same time. On another embodiment, the compound of the invention and at least one known cancer chemotherapeutic agent are administered according to their individual dose schedule, so long as the compounds reach therapeutic levels in the blood.

In particular embodiments, (4-Methoxy-phenyl)-methyl-(2-methyl-quinazolin-4-yl)-amine hydrochloride is administered with bevacizumab. For example, bevacizumab may be administered at a dose of not more than about 25 mg/kg, such as from about 10 mg/kg to about 15 mg/kg before, after or concurrently with administration of (4-Methoxy-phenyl)-methyl-(2-methyl-quinazolin-4-yl)-amine hydrochloride. In specific embodiments (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with bevacizumab administered at a dose of not more than about 25 mg/kg, such as from about 10 mg/kg to about 15 mg/kg.

In particular embodiments, (4-Methoxy-phenyl)-methyl-(2-methyl-quinazolin-4-yl)-amine hydrochloride is administered with oxaliplatin. For example, oxaliplatin may be administered at a dose of not more than about 200 mg/m² or not more than about 85 mg/m², such as from about 55 mg/m² to about 85 mg/m² before, after or concurrently with administration of (4-Methoxy-phenyl)-methyl-(2-methyl-quinazolin-4-yl)-amine hydrochloride. In specific embodiments (4-Methoxy-phenyl)-methyl-(2-methyl-quinazolin-4-yl)-amine hydrochloride is administered as therapy at a dose of not more than about 4.5 mg/m², such as not more than 3.3 mg/m² or not more than about 2.1 mg/m² in combination with oxaliplatin administered at a dose of not more than about 200 mg/m² or not more than about 85 mg/m², such as from about 55 mg/m² to about 85 mg/m².

In one embodiment, the invention provides a method of reducing the size or slowing the growth of neoplasms. Reductions in size and/or growth of neoplasms may be measured by the Response Evaluation Criteria in Solid Tumors (RECIST) Guidelines (see Therasse et al. J. Nat. Cancer Institute 92:205-216 (2000), herein incorporated by reference in its entirety). For example, the method may reduce the average size of lesions in patients by about 30% or more as measured at four weeks post-treatment by identifying up to 5 lesions per organ and 10 lesions in total, and determining the reduction in length at the longest diameter of the lesion. In yet another embodiment, the invention provides a method for improving the survival of patients with or at risk of forming tumors.

Compounds used in practicing the present invention can be prepared by a variety of art known procedures. For example, in practicing the present invention, (4-Methoxy-phenyl)-methyl-(2-methyl-quinazolin-4-yl)-amine hydrochloride may be prepared using methods known to those skilled in the art. Specifically, (4-Methoxy-phenyl)-methyl-(2-methyl-quinazolin-4-yl)-amine hydrochloride may be prepared according to International Pat. Publication No. WO 2005/003100 and as illustrated by the exemplary reaction in Scheme 1.

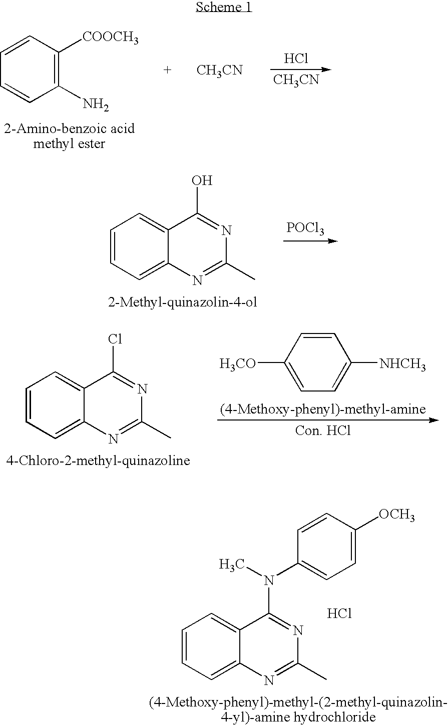

In addition, many of the compounds are commercially available from a variety of sources. For example, carboplatin is available from Bristol-Myers Squibb Company (New York, N.Y.), oxaliplatin is available from Sanofi-Aventis (Paris, France), and temozolomide is available from Schering-Plough (Kenilworth, N.J.). Avastin® (bevacizumab) is available from Genentech (San Francisco, Calif.), Sutent® (sunitinib) is available from Pfizer (New York, N.Y.), and Nexavar® (sorafenib) is available from Bayer (Leverkusen, Germany).

The therapeutic methods of present invention also include methods comprising administering to an animal an effective amount of a compound, or a pharmaceutically acceptable salt, acid or base of (4-Methoxy-phenyl)-methyl-(2-methyl-quinazolin-4-yl)-amine hydrochloride. In one embodiment, a pharmaceutical composition comprising (4-Methoxy-phenyl)-methyl-(2-methyl-quinazolin-4-yl)-amine hydrochloride, or a pharmaceutically acceptable salt, acid, or base of said compound, in combination with a pharmaceutically acceptable vehicle is administered. Examples of pharmaceutically acceptable addition salts for (4-Methoxy-phenyl)-methyl-(2-methyl-quinazolin-4-yl)-amine hydrochloride, (or base thereof) include inorganic and organic acid addition salts, such as hydrochloride, hydrobromide, phosphate, sulphate, citrate, lactate, tartrate, maleate, fumarate, mandelate and oxalate; and inorganic and organic base addition salts with bases, such as sodium hydroxy, Tris(hydroxymethyl)aminomethane (TRIS, tromethane) and N-methyl-glucamine. The present invention also includes methods comprising administering to an animal an effective amount of (4-Methoxy-phenyl)-methyl-(2-methyl-quinazolin-4-yl)-amine hydrochloride, or a pharmaceutically acceptable salt or prodrug thereof, and one or more liquid diluents. Such compositions include compositions disclosed in PCT Pub. No. WO 2006/138608, and may be manufactured according to the methods disclosed therein, the relevant portions of which are incorporated herein by reference.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the compounds of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### Example 1

### Preparation of (4-Methoxy-phenyl)-methyl-(2-methyl-quinazolin-4-yl)-amine hydrochloride

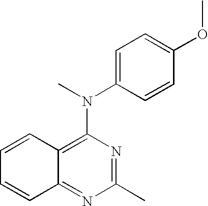

### (4-Methoxy-phenyl)-methyl-(2-methyl-quinazolin-4-yl)-amine hydrochloride

a) 4-Chloro-2-methyl-quinazoline: A stirred suspension of 2-methyl-4(3H)-quinazolinone (5 g, 31.2 mmol) in POCl₃ (100 mL) was heated at 120° C. for 3 h. The excess POCl₃ was removed under vacuum, then to the residue was added crushed ice and 200 mL of saturated NaHCO₃, and the mixture was extracted with ethyl acetate (200 mL×2). The combined extracts were washed with water, saturated NaCl, dried over anhydrous MgSO₄, filtered and concentrated. The crude product was purified by column chromatography (5-8% ethyl acetate/hexane) to give the title compound (2.5 g, 14.0 mmol, 45%). ¹H NMR (CDCl₃): 8.21-8.25 (m, 1H), 7.89-7.99 (m, 2H), 7.66 (ddd, 1H, J=1.8, 6.6, 8.7), 2.87 (s, 3H).

b) (4-Methoxy-phenyl)-methyl-(2-methyl-quinazolin-4-yl)-amine hydrochloride: The title compound was prepared from 4-chloro-2-methyl-quinazoline (2.31 g, 12.9 mmol) and (4-methoxy phenyl)-methyl-amine (2.0 g, 14.6 mmol) by a procedure similar to example 1b and was isolated as solids (2.90 g, 9.18 mmol, 71%). ¹H NMR (CDCl₃): 8.53 (dd, 1H, J=0.6, 8.1), 7.7 (ddd, 1H, J=1.2, 7.2, 8.4), 7.22 (m, 2H), 7.13 (ddd, 1H, J=1.2, 7.2, 8.7), 7.05 (m, 2H), 6.76 (d, 1H, J=8.7), 3.91 (s, 3H), 3.78 (s, 3H), 2.96 (s, 3H).

### Example 2

### Pharmaceutical Composition

A pharmaceutical composition is prepared by combining and mixing 100 grams of (4-Methoxy-phenyl)-methyl-(2-methyl-quinazolin-4-yl)-amine hydrochloride and 1 gram of BHT and dissolving into 10 liters of D5W with the pH adjusted to pH=5 with hydrochloric acid. This solution is sterile filtered using a 0.2 μm Teflon filter (PTFE).

### Example 3

### Pharmaceutical Composition

A pharmaceutical composition was formed by dissolving 300.1 grams (4-Methoxy-phenyl)-methyl-(2-methyl-quinazolin-4-yl)-amine hydrochloride into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 4

### Pharmaceutical Composition

A pharmaceutical composition was formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 5

### Pharmaceutical Composition

A pharmaceutical composition is formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 11.652 kg viscosity reducing agent (ethanol 190 proof), and 2 kg WFI (water for injection). This solution is sterile filtered through a 0.2 μm Millipore Durapore filter (PVDF), and packaged into 10 ml sterile glass vials.

### Example 6

### Method of Administration

About 0.01 ml to about 50 ml of the pharmaceutical composition of Example 5 is accurately measured and then added to an i.v. bag containing about 100 ml to about 1000 ml of sterile dextrose 5% in water (D5W). The amount of pharmaceutical composition and D5W used varies according to the desired therapeutic dose and size of the patient. The resulting mixture is then parenterally infused into the patient.

### Example 7

### Vascular Disruption Effects of (4-Methoxy-phenyl)-methyl-(2-methyl-quinazolin-4-yl)-amine hydrochloride

(4-Methoxy-phenyl)-methyl-(2-methyl-quinazolin-4-yl)-amine hydrochloride was tested in a human tumor xenograft model to determine its vascular disruption effects. Crl:Nu/Nu-nuBR mice were grown and injected with 1×107 human ovarian OVCAR-3 carcinoma cells on the right flank and allowed to grow to various tumor volumes. Mice were then dosed with either vehicle, 100 mg/kg combretastatin A-4 phosphate (CA4P) (obtained from Toronto Research Chemicals, Ontario, Canada) or 10 mg/kg (4-Methoxy-phenyl)-methyl-(2-methyl-quinazolin-4-yl)-amine hydrochloride.

Animals were housed by groups in Positive Individual Ventilation (PIV) cages in flat-bottom cages (Thoren Caging Systems, Hazelton, Pa.) with no more than ten mice per cage. Cages contained autoclaved TEK-Fresh bedding (Harlan, Indianapolis, Ind.), which was changed every seven days. Environmental controls were set to maintain a temperature between 65° F. and 75° F. with a relative humidity of 30-70% in a 12:12 hour light:dark cycle. Animals were feed gamma-irradiated 2019 rodent chow ad libitum (Harlan, Indianapolis, Ind.). Tap water was sterilized using manufacture recommended conditions and supplied via an automated watering system ad libitum (Edstrom Industries, Waterford, Wis.). Twenty four hours after dosing, mice were sacrificed and tumors and hearts removed, fixed, sectioned and stained with hematoxylin and eosin Y (each obtained from Richard Allen Scientific, Kalamazoo, Mich.).

Examination of all tumors revealed that necrosis was a prominent feature. Additionally, the tumors were highly anaplastic with marked pleomorphism and high mitotic indices. Within vehicle or compound treated cohorts, there was little variation in tumor morphology from animal to animal. This tumor was naturally arranged in packets surrounded by a thin fibrous stroma.

Rapid tumor proliferation often results in central necrosis of individual packets because the tumor outgrows the vascular supply due to rapid proliferation. In the vehicle control, this process appeared to be responsible for the central necrosis observed in some tumor nodules. Also, tumor tissue from the vehicle control contained large cystic areas suggestive of germinal follicles.

In tumors from mice treated with either (4-Methoxy-phenyl)-methyl-(2-methyl-quinazolin-4-yl)-amine hydrochloride or CA4P, the necrosis appeared to originate from a different process than rapid proliferation. Instead of necrosis of individual tumor nodules, the necrosis was widespread and involved the supporting stroma, as well as the neoplasm. Blood vessels in the necrotic areas were consistently congested with perivascular hemorrhage. This pattern of necrosis suggests that the blood supply to the tumors was disrupted at some point. Unlike the vehicle control, the central area of the tumors treated with either (4-Methoxy-phenyl)-methyl-(2-methyl-quinazolin-4-yl)-amine hydrochloride or CA4P had extensive necrosis with a rim of viable tumor tissue around the periphery. Overall, the degree of necrosis appeared greater in the (4-Methoxy-phenyl)-methyl-(2-methyl-quinazolin-4-yl)-amine hydrochloride treated animals than in the CA4P treated animals.

### Example 8

### Phase I Clinical Trial of Administration of (4-Methoxy-phenyl)-methyl-(2-methyl-quinazolin-4-yl)-amine hydrochloride for Subjects with Refractory Solid Tumors

An open-label, dose-escalating, multiple-dose study to define the safety, tolerability and pharmacokinetics of weekly intravenous administration of (4-Methoxy-phenyl)-methyl-(2-methyl-quinazolin-4-yl)-amine hydrochloride was performed. A dosing schedule (each 4 week cycle) was performed for (4-Methoxy-phenyl)-methyl-(2-methyl-quinazolin-4-yl)-amine hydrochloride weekly for 3 weeks with no infusion on the fourth week of each cycle. Subjects with refractory solid tumors were enrolled in cohorts of 3. During Cycle 1, subjects were hospitalized during each infusion of (4-Methoxy-phenyl)-methyl-(2-methyl-quinazolin-4-yl)-amine hydrochloride and remained for observation and safety evaluation for approximately 24 hours following the end of the infusion. All subject had continuous telemetry for 2 hours prior to infusion, for 1-2 hour infusion and for 3 hours after the end of the infusion. Any clinically significant electrocardiographic (ECG) wave form abnormality was recorded and prolongation of the monitoring period extended at the discretion of the principal investigator.

Electrocardiograms were obtained prior to starting the infusion and within 30 minutes of the end of infusion for each infusino of the first cycle. Electrocardiograms on Day 1 were obtained in triplicate 5 minutes apart.

Neurocognitive assessments were made by administration of the Mini-Mental State Examination (MMSE), the Hopkins Verbal Learning and timed Grooved Pegboard tests before administration of the intravenous infusion and approximately 24 hours of the infusion at each weekly administration of the first cycle.

On days 1, 8, and 15 of each cycle, vital signs were obtained prior to the first dose, at 15, 30, and 60 minutes after the initiation of the infusion, and at 0.5, 1, 1.5, 2, and 4 hours after the end of the intravenous infusion. Vital signs at all time points beyond the start of the intravenous infusion included heart rate, blood pressure and respirations. Temperature was measured at the end of the infusion and 4 hours later.

Individual subjects were allowed to continue on repeated weekly ×3 administrations every 28 days with no dose increase provided there was no unacceptable toxicity or disease progression.

Tumor response was evaluated by response evaluation criteria in solid tumors (RECIST) criteria. To prevent sever hypersensitivity reactions due to Cremophor® EL, subjects were premedicated with oral dexamethasone (20 mg) administered approximately 12 and 6 hours before the intervenous infusion with (4-Methoxy-phenyl)-methyl-(2-methyl-quinazolin-4-yl)-amine hydrochloride, diphenhydramine (50 mg) or its equivalent administered intravenously 30-60 minutes before (4-Methoxy-phenyl)-methyl-(2-methyl-quinazolin-4-yl)-amine hydrochloride, and cimetidine (300 mg) or ranitidine (50 mg) administered intravenously 30-60 minutes before (4-Methoxy-phenyl)-methyl-(2-methyl-quinazolin-4-yl)-amine hydrochloride.

Dose escalation of subjects proceeded sequentially as presented in Table 1 below:

TABLE 1

| Cohort Number | Dose level (modified Fibonacci series) |
|---|---|
| Cohort 1 | Dose 1 = 0.3 mg/m² |
| Cohort 2 | Dose 2 = 0.6 mg/m² |
| Cohort 3 | Dose 3 = 1.0 mg/m² |
| Cohort 4 | Dose 4 = 1.5 mg/m² |
| Cohort 5 | Dose 5 = 2.1 mg/m² |
| Cohort 6 | Dose 6 = 2.7 mg/m² |
| Cohort 7 | Dose 7 = 3.3 mg/m² |

The results of the Phase 1 Trial show that there is no evidence of cytotoxity peripherally at the administered doses. There were incidences of intratumor bleeding and the dose limiting toxicity was demonstrated to be vascular in nature, manifested by an acute coronary syndrome. There were no significant effects on cardiac conduction (PR, QRS or QTC) but there was a dose-related increase in systolic blood pressure and occasional episodes of bradycardia. Accordingly, (4-Methoxy-phenyl)-methyl-(2-methyl-quinazolin-4-yl)-amine hydrochloride is thus shown to be safe and tolerable.

### Example 9

### Anti-tumor Effect of (4-Methoxy-phenyl)-methyl-(2-methyl-quinazolin-4-yl)-amine hydrochloride and Sutent®

Crl:Nu/Nu-nuBR mice are obtained from Charles River Labs, Wilmington, Mass. and implanted subcutaneously (s.c.) in the right flank with 1×107 human ovarian OVCAR-3 carcinoma cells that are allowed to grow to tumor volumes averaging 500 mm³. Mice are segregated into groups of 10 and then dosed with either vehicle, (4-Methoxy-phenyl)-methyl-(2-methyl-quinazolin-4-yl)-amine hydrochloride (5 mg/kg, single dose), Sutent® (40 mg/kg daily) (available from Pfizer®, New York, N.Y.), or the combination of (4-Methoxy-phenyl)-methyl-(2-methyl-quinazolin-4-yl)-amine hydrochloride (5 mg/kg, single dose) and Sutent® (40 mg/kg daily). At various times after dosing, selected animals are sacrificed and tumors and kidneys removed, fixed, sectioned and stained with hematoxylin and eosin Y.

Animals are housed by groups in Positive Individual Ventilation (PIV) cages in flat-bottom cages (Thoren Caging Systems, Hazelton, Pa.) with no more than ten mice per cage. Cages contain autoclaved TEK-Fresh bedding (Harlan, Indianapolis, Ind.), which is changed every seven days. Environmental controls are set to maintain a temperature between 65° F. and 75° F. with a relative humidity of 30-70% in a 12:12 hour light:dark cycle. Animals are feed gamma-irradiated 2019 rodent chow ad libitum (Harlan, Indianapolis, Ind.). Tap water is sterilized using manufacture recommended conditions and supplied via an automated watering system ad libitum (Edstrom Industries, Waterford, Wis.). The mice are observed daily for mortality and signs of toxicity. Tumors and body weights are measured from Days 1 to end of study with the frequency determined by the rate of growth of the tumor. Average tumor sizes are calculated using Microsoft Excel 2000 (Microsoft; Redmond, Wash.) to determine the effect of test compounds on tumor size. Values are then transferred to Prism software for graphing. Statistical analysis of variance with unadjusted pair wise comparison is performed using SAS software (SAS; Cary, N.C.).

### Example 10

### Determination of the Effect on Angiogenesis of Agents

The tumor vascular window model and vascular length density analysis is used to determine the effect on angiogenesis of agents. C57BL/6 mice receive subcutaneous injections in the right thigh with 106 viable cells of a murine lung carcinoma (LLC) suspended in 0.2 ml of a 1:1 solution of Matrigel. A solution of penicillin-streptomycin (200 p. 1) is injected into the hind limb of the mouse before the procedure. The dorsal skin-fold window is a 3-g plastic frame applied to the skin of the mouse that remains attached for the duration of the study. The chamber is screwed together and the epidermis is incised and remain open with a plastic covering. The midline is found along the back, and a clip is placed to hold the skin in position. A template, equivalent to the outer diameter of the chamber, is traced, producing the outline of the incision. A circular cut is made tracing the perimeter (7-mm diameter) of the outline followed by a crisscross cut, thereby producing four skin flaps. The epidermis of the four flaps is then removed using a scalpel with an effort to follow the hypodermis superior to the fascia. The area is then trimmed with fine forceps and iris scissors. The template is removed and the top piece of the chamber is fixed with screws. During surgery, the area is kept moist by applying moist drops of PBS with 1% penicillin/streptomycin solution. The bottom portion of the chamber is put in place, and the top is carefully positioned on the cut side so that the window and the circular incision are fitted. Antibiotic ointment is applied at this time. The three screws that held the chamber together are then positioned into the chamber holes and tightened so that the skin is not pinched, thus avoiding diminished circulation. Tumor blood vessels develop in the window within 1 week.

The time- and dose-dependent response of tumor blood vessels to agents are studied using the window model. Mice (N=5) are studied in each of the treatment groups. The window frame is marked with coordinates, which are used to photograph the same microscopic field each day. Vascular windows are photographed using ×4 objective to obtain a ×40 total magnification. Color photographs are used to catalogue the appearance of blood vessels on days 0-7. Photographs are scanned into Photoshop software, and vascular center lines are positioned by ImagePro Software and verified by an observer blinded to the treatment groups. Tumor blood vessels are quantified by the use of ImagePro software, which quantifies the vascular length density of blood vessel within the microscopic field. Center lines are verified before summation of the vascular length density. The mean and 95% confidence intervals of vascular length density for each treatment group are calculated, and variance is analyzed by the general linear models and Bonferroni t test.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of treating cancer in a mammal in need of such treatment, comprising administering to the mammal an effective amount of (4-Methoxy-phenyl)-methyl-(2-methyl-quinazolin-4-yl)-amine, or a pharmaceutically acceptable salt thereof, and an effective amount of one or more chemotherapeutic agents chosen from antiangiogenic agents and cytotoxic agents.

2. The method of claim 1, wherein the pharmaceutically acceptable salt is (4-Methoxy-phenyl)-methyl-(2-methyl-quinazolin-4-yl)-amine hydrochloride.

3. The method of claim 2, wherein the effective amount of (4-Methoxy-phenyl)-methyl-(2-methyl-quinazolin-4-yl)-amine hydrochloride is administered at a dose of not more than about 4.5 mg/m².

4. The method of claim 2, wherein the effective amount of (4-Methoxy-phenyl)-methyl-(2-methyl-quinazolin-4-yl)-amine hydrochloride is administered at a dose of not more than about 3.3 mg/m².

5. The method of claim 2, wherein the effective amount of (4-Methoxy-phenyl)-methyl-(2-methyl-quinazolin-4-yl)-amine hydrochloride is administered at a dose of not more than about 2.7 mg/m².

6. The method of claim 2, wherein the effective amount of (4-Methoxy-phenyl)-methyl-(2-methyl-quinazolin-4-yl)-amine hydrochloride is administered at a dose of not more than about 2.1 mg/m².

7. The method of claim 2, wherein the effective amount of (4-Methoxy-phenyl)-methyl-(2-methyl-quinazolin-4-yl)-amine hydrochloride is administered at a dose of not more than about 1.5 mg/m².

8. The method of claim 2, wherein the effective amount of (4-Methoxy-phenyl)-methyl-(2-methyl-quinazolin-4-yl)-amine hydrochloride is administered at a dose of not more than about 0.5 mg/m².

9. The method of claim 1, wherein the chemotherapeutic agent is an antiangiogenesis agent.

10. The method of claim 9, wherein the antiangiogenesis agent is chosen from penicillamine, Tetrathiomolybdate, trientine, British Anti-Lewisite, dimercaptosuccinic acid, clioquinol, pyrrolidine dithiocarbamate, alpha-lipoic acid, L-taurine, pyrrolidine dithiocarbamate, an NSAID, and brucillamine.

11. The method of claim 9, wherein the antiangiogenesis agent is chosen from bevacizumab, sunitinib, sorafenib, vatalanib, semaxanib, ZD6474, SU6668, AG-013736, AZD2171, and AEE788.

12. The method of claim 9, wherein the antioangiogenesis agent is bevacizumab.

13. The method of claim 12, wherein the effective amount of bevacizumab is administered at a dose of not more than about 25 mg/m².

14. The method of claim 12, wherein
(a) (4-Methoxy-phenyl)-methyl-(2-methyl-quinazolin-4-yl)-amine hydrochloride is administered at a dose of between about 2.1 mg/m² and about 3.3 mg/m², and
(b) bevacizumab is administered at a dose of between about 10 mg/kg and about 15 mg/kg.

15. The method of claim 1, wherein the chemotherapeutic agent is chosen from temozolomide, dacarbazine, BCNU, CCNU, vinorelbine, teniposide, irinotecan, daunomycin, idarubicin, cytarabine, gemcitibine, capecitibine, carboplatin, and oxaliplatin.

16. The method of claim 1, wherein the chemotherapeutic agent is carboplatin and wherein the effective amount of carboplatin is administered at a dose that provides the subject an AUC of not more than about 6 mg/mL (min).

17. The method of claim 1, wherein
(a) (4-Methoxy-phenyl)-methyl-(2-methyl-quinazolin-4-yl)-amine hydrochloride is administered at a dose of between about 2.1 mg/m² and about 3.3 mg/m², and
(b) carboplatin is administered at a dose that provides the subject an AUC of between about 4 mg/mL (min) and about 6 mg/mL (min).

18. A unitary pharmaceutical composition comprising:
a pharmaceutically acceptable carrier;
an effective amount of (4-Methoxy-phenyl)-methyl-(2-methyl-quinazolin-4-yl)-amine, or a pharmaceutically acceptable salt thereof; and
one or more chemotherapeutic agents chosen from antiangiogenic agents and cytotoxic agents.

19. The unitary pharmaceutical composition of claim 18, wherein the antiangiogenesis agent is chosen from penicillamine, Tetrathiomolybdate, trientine, British Anti-Lewisite, dimercaptosuccinic acid, clioquinol, pyrrolidine dithiocarbamate, alpha-lipoic acid, L-taurine, pyrrolidine dithiocarbamate, brucillamine, and NSAIDs.

20. The unitary pharmaceutical composition of claim 18, wherein the chemotherapeutic agent is chosen from temozolomide, dacarbazine, BCNU, CCNU, vinorelbine, teniposide, irinotecan, daunomycin, idarubicin, cytarabine, gemcitibine, capecitibine, carboplatin, and oxaliplatin.
